(19) United States
(12) Patent Application Publication
(10) Pub. No.: US 2008/0311053 A1
(43) Pub. Date: Dec. 18, 2008

(54) Fluorinated phosphate ester surfactant and fluorinated alcohol compositions

(75) Inventor: Michael Allen Curtis, Glen Rock, NJ (US)

Correspondence Address: Irving M. Fishman, 89 Headquarters Plaza, Suite 1422, North Tower, Morristown, NJ, 07960, US

(73) Assignee: Mason Chemical Company

(21) Appl. No.: 12/157,618

(22) Filed: Jun. 12, 2008

Related U.S. Application Data
(60) Provisional application No. 60/934,632, filed on Jun. 14, 2007.

(51) Int. Cl. A61K 9/12

(52) U.S. Cl. 424/47

(57) ABSTRACT

Foaming compositions having high solvent content and compositions for foaming high solvent content formulations are disclosed having a fluorinated phosphate ester surfactant (“FPES”) of the formula (R^{a}—O)ₓ—P(O)—(OR^{b})_{y} in combination with a fluorinated alcohol (“FA”) of the formula R^{c}OH, where x is 1 to 3, y is (3−x), R^{a} is a group R¹-R²- with R¹ being a fluorinated C₆₋₁₈hydrocarbon optionally interrupted by groups of one or two non-fluorinated (or less fluorinated) carbons per interruption, and/or optionally interrupted by heteroatoms or functional groups, and R^{b} is selected from H and positively charged amines, and R^{c} is the same or different as R^{a} and if different is selected from the same group of R^{a} materials as well as from analogous groups to R^{a} except that they have only 1-3 carbons in the R¹ portion of R^{a} instead of the R^{a} requirement of at least 4 carbons. The ratio of FA to FPES is at least 5% FA to not more than 95% FPES based on the total FA+FPES weight. The compositions are especially useful for foaming high organic solvent content (>20% by weight solvent) with the solvent being selected from lower aliphatic alcohol, C₅₋₆cyclic saturated or unsaturated alcohols, lower alkyl-C(O)-lower alkyl, lower alkyl-O—C(O)-lower alkyl, lower alkylene glycols, and lower alkylene glycol-mono lower alkyl ethers.

### CROSS-REFERENCE TO RELATED APPLICATIONS

This application claims benefit of U.S. provisional application Ser. No. 60/934,632, filed Jun. 14, 2007.

### STATEMENT REGARDING FEDERALLY SPONSORED RESEARCH OR DEVELOPMENT

Not Applicable

### FIELD OF THE INVENTION

The present invention relates to foamable formulations and compositions for foaming formulations where the formulation contains a high level of organic solvent, especially a high level of lower alcohol. The invention further relates to the use of fluorinated phosphate ester surfactant (“FPES”) of the formula (R^{a}—O)—P(O)—(OR^{b})_{y} in combination with a fluorinated alcohol (“FA”) of the formula R^{a}OH. The invention further relates to the use of such compositions and formulations in the personal care product arena, typically foamable hand sanitizers, foamable shampoos, foamable personal cleansers (such as shower or bath oil foams, and other foamable soaps), foamable brushless shave creams, sunscreen foams, foam make-up removers, foamable nail polish removers, foam analgesic rubs, antiperspirant foams, foam pet grooming products, etc. In addition, the invention relates to medicated foams containing any number of suitably dispensed active agents, typically for external use, but also potentially for internal use (generally in body cavities other than oral, but oral use may be suitable as well). Still further, the present invention relates to use of the compositions in household and industrial (hard surface or non-hard-surface) cleaning compositions. The invention further relates to use thereof in foaming compositions used in industrial fields such as oilfield, foam insulation, foam containers, and foam packaging products, to name a few. Those of ordinary skill in the art will appreciate yet other foam applications of the present invention.

### BACKGROUND OF THE INVENTION

Ethanol and/or isopropyl alcohol compositions with at least 60% percent v/v (approximately 52% by weight) are well known to be antibacterial and are therefore widely accepted for disinfecting purposes. Nonetheless due to the inherent characteristics of alcohol, it is perceived that the higher the alcohol content the better the product and a higher than 60% by volume alcohol content solution is more desirable.

Alcohol disinfectant solutions are generally thickened in order to eliminate waste and facilitate spreading the composition throughout the desired area. It is also known that other than gelling agents one can use paraffin or waxes to achieve thickening of a solution with high alcohol concentration. One of the disadvantages of gels and such thickened alcohol containing compositions is that they generally leave a tacky feeling on the hands after one use, and even if not with one use, the effect tends to build with multiple use in the day, making it necessary to eventually wash off the thickeners before continuing the usage of such an alcohol antiseptic solution. The present invention alcoholic disinfectant formulations do not contain such thickeners, do not leave such an undesirable feel, and therefore do not need to be washed off after having been used repeatedly. Recently, US 2005-0129626 has proposed formulations for foaming high alcoholic content disinfectant compositions that also avoid the use of such thickeners by employing certain surfactants, among which are compounds of the formula

(F(CF₂CF₂)ᵣ—CH₂CH₂O)ₛP(O)—(O^{-+NH}₄)ₜ

where s=1 or 2, t=2 or 1, and r=1 to 7, or mixtures thereof. US 2005-0129626 indicates that stable foams are produced and gel-like thick compositions can be formed without the need for thickeners of the prior art. Unfortunately, these compositions produce foams that are still insufficiently stable and still have a tacky feel (although reduced as compared to those formulations that include the thickeners as stated therein). The present invention provides much more stable foams than those set forth in US 2005-0129626.

In the few “foamable” high alcohol content products disclosed prior to US 2005-0129626, the types of foam obtained were not similar to those expected from aqueous solutions. The foams obtained are described as fast or aerated foam, quick breaking, with low or limited stability, which would not last for more than one minute, being generally gone within seconds. US 2005-0129626 alleges to have produced longer lasting foams with the surfactants described therein, yet these foams are still not very stable and tend to break much too quickly (although not as fast as the foams of the earlier prior art). The present invention significantly improves the foam stability and other foam characteristics.

It has been disclosed that fluorosurfactants and alcohol can be combined to produce a “stable” foam by a process using a high-pressurized means to generate the foam. Highly stable pressurized foams containing high lower alcohol contents and methods of forming and using such pressurized foams in the oil industry using a non-ionic surfactant or mixture of non-ionic surfactants of a specific group of fluorosurfactants are provided in U.S. Pat. No. 4,440,653. The compositions in this patent require the use of a pressurized gas system to generate the foam. In contrast, high pressurization is not required to achieve the stable foams of the present invention. Other recent references relating to foaming alcoholic solutions include US 2006-0182690 and US 2006-0104919.

### OBJECTS OF THE INVENTION

It is therefore an object of the present invention to provide a method for stably foaming a composition of a high organic solvent content.

It is another object of the invention to provide a concentrate for use in stably foaming a high organic solvent content formulation.

It is still another object of the invention to provide a composition having a high organic solvent content that can be stably foamed.

It is yet another object of the invention to provide method of stably foaming a composition having a high organic solvent content without the use of thickeners.

It is an even further object of the invention to provide method of stably foaming a composition having a high organic solvent content without the use of foam stabilizers.

It is yet another object of the invention to provide a stable foam composition having a high organic solvent content.

It is still another object of the invention to provide a stable foam composition having a high organic solvent content in the absence of a thickener.

It is an even further object of the invention to provide a stable foam composition having a high organic solvent content in the absence of a foam stabilizer.

It is still a further object of the invention to provide a personal care product within one or more of the foregoing objects.

Yet another object of the invention is to provide a household cleanser product within one or more of the foregoing objects.

Still another object of the invention is to provide an industrial cleanser product within one or more of the foregoing objects.

Yet another object of the invention is to provide a product for production of foamed articles of manufacture.

Another object of the invention is to provide a foamable formulation for use in oil and/or natural gas recovery from geologic sites.

Still further objects of the invention will be apparent to those of ordinary skill in the art upon reading the following description and claims.

### BRIEF SUMMARY OF THE INVENTION

These and other objects of the invention are surprisingly achieved utilizing in conjunction with a high organic co-solvent composition or inclusion into said composition of a blend of a fluorinated phosphate ester surfactant (“FPES”) of the formula (R^{a}—O)ₓ—P(O)—(OR^{b})_{y} in combination with a fluorinated alcohol (“FA”) of the formula R^{c}OH, where x is 1 to 3, y is (3−x), R^{a} is a group R¹-R²- with R¹ being a fluorinated C₆₋₁₈hydrocarbon optionally interrupted by groups of one or two non-fluorinated (or less fluorinated than in the rest of R¹ exclusive of the optional interruptions) carbons per interruption, and/or optionally interrupted by heteroatoms or functional groups, R¹ being an alkyl chain of 1-3 carbons, and R^{b} is selected from H, cations (such as, without limitations, alkali metal, alkaline earth metal, and mono or polyvalent cations) and positively charged amines, and R^{c} is, independently of R^{a}, selected from the same group as R^{a} or may also be a shorter carbon chain version thereof. The ratio of FA to FPES is from (a) at least about 5% w/w FA: not more than about 95% w/w FPES to (b) generally not more than about 90% w/w FA: about 10% w/w FPES (i.e., about 1- about 171 parts by weight FA to 19 parts by weight FPES). The compositions are especially useful for foaming high organic solvent content (at least about 20% by weight solvent) with the solvent being selected from lower aliphatic alcohol, C₅₋₆cyclic saturated or unsaturated alcohols, lower alkyl-C(O)-lower alkyl, lower alkyl-O—C(O)-lower alkyl, lower alkylene glycols, and lower alkylene glycol-mono lower alkyl ethers.

### BRIEF DESCRIPTION OF THE DRAWING

Not applicable

### DETAILED DESCRIPTION OF THE INVENTION

The present invention is directed to compositions and methods for obtaining stable foams of high organic solvent content materials, as well as formulations which include both the high organic solvent and the materials which make the composition foamable into a stable foam.

In all discussions, unless specifically set forth otherwise or clearly apparent from the context, the term “lower” shall mean a carbon chain of from 1-6 carbons, preferably 1-4 carbons.

The organic solvents which are present in high % and are rendered stably foamable by the invention process and formulations are selected from lower aliphatic alcohol (preferably C₁₋₄alkanol; particularly preferably methanol, ethanol, n-propanol, iso-propanol, n-butanol, sec-butanol, and tertiary-butanol; more preferably ethanol and isopropanol); C₅₋₆cyclic saturated or unsaturated alcohols (including cyclohexanol, cyclohexenol, etc.); 5-6 membered cyclic saturated or unsaturated solvents having an oxygen ring member (such as furan, tetrahydrofuran, pyran, dihydropyran, and tetrahydropyran); lower alkyl-C(O)-lower alkyl (such as acetone, methyl ethyl ketone, diethyl ketone, etc); lower alkyl-O—C(O)-lower alkyl (such as methyl acetate, methyl n-propionate, methyl isopropionate, ethyl acetate, ethyl n-propionate, ethyl isopropionate, n-propyl acetate, n-propyl n-propionate, isopropyl acetate, isopropyl isopropionate, etc.), lower alkylene glycols (such as ethylene glycol, 1,2-propylene glycol and 1,3-propylene glycol), and lower alkylene glycol-mono lower alkyl ethers (such as ethylene glycol mono methyl ether, ethylene glycol mono ethyl ether, ethylene glycol mono propyl ethers, and ethylene glycol mono butyl ethers, etc.). The lower alkyl alcohols are preferred, especially ethanol and isopropanol for the personal care product market products. (Acetone is also a preferred solvent in such personal care products as nail polish removers etc. Methanol is a preferred solvent in applications involving oilfields.) These or other solvents will be preferred for other applications as will be apparent to those of ordinary skill in the art of the particular end use.

The foregoing organic solvents, whether as part of other products to which the stable foam imparting components to be described below are added, or as a component added with or added separately from the stable foam imparting components, comprise at least about 20% by weight of the composition that will result in the foam, preferably at least about 25% by weight, more preferably at least about 30% by weight, still more preferably at least about 35% by weight, even more preferably at least about 40% by weight. Highly preferred specific embodiments of the foamable alcohols contain 50% or more, 60% or more, or even 70% or more alcohol. Thus, a formulation that has no such organic solvent can be made stably foamable with the present invention by addition of the appropriate solvent as set forth above and the stable foam imparting components to be discussed below. Alternatively, a formulation having one or more of the above solvents in amounts (in the aggregate) in the above described amounts can be rendered stably foamable by mere addition of the stable foam imparting components alone or with additional such solvent. Further, a composition having some, but not all of the stable foam imparting components required can be rendered stably foamable by addition of the remaining component(s) required by the present invention.

To make the high organic solvent content formulations stably foamable, the following stable foam imparting components need to be present, and they can be added separately, or in various combinations. Thus, compositions having various blends of the stable foam imparting components (or some, but not all, of them) are also contemplated as part of the present invention, especially for use in completing the present invention stably foamable compositions. This includes concentrates for addition to otherwise complete formulations. The stable foam imparting components include at least two distinct components in a particular range of ratios. The first component is a fluorinated phosphate ester surfactant (“FPES”) of Formula I

(R^{a}—O)ₓ—P(O)—(O^{-+R}^{b})_{y} I

in combination with a second component which is a fluorinated alcohol (“FA”) of Formula II

R^{c}OH II,

where x is 1 to 3, y is (3−x), R^{a} is a group R¹-R²- with R¹ being a fluorinated C₆₋₁₈hydrocarbon (preferably a substantially fluorocarbon, more preferably a perfluorcarbon) optionally interrupted by groups of one or two non-fluorinated (or less fluorinated) carbons (such as —CHF— and —CH₂—) per interruption, and/or optionally interrupted by one or two heteroatoms (preferably —O—, —S—, or —NH—, but not two adjacent atoms of the same kind) or one functional group (preferably —C(O)O—, —OC(O)—, —C(S)O—, —OC(S)—, —SC(S)—, —C(S)S—, —S(O₂)—, —S(O₂)NH—, or —NHS(O₂)—, etc.) per interruption, R² is an alkyl chain of 1-3 carbons selected from —CH₂—, —CH₂CH₂—, —CH₂CH₂CH₂—, and —CH₂(CH₃)CH₂—, preferably —CH₂CH₂—; R^{c} is, independently of R^{a}, selected from same members as R^{a} and also from corresponding groups that are the same as R^{a} groups except that they are of shorter carbon lengths in the R¹ portion thereof in the range of 1-5 carbons; and R^{b} is selected from H, alkali metal (lithium, sodium, or potassium, preferably sodium), and positively charged amines (such as those of Formula III.

(H)ᵥ⁺N(R³R⁴)₄₋ᵥ III

where v is an integer of 1 to 4 and each R³ is independently selected from a straight or branched chain of 1-18 carbon atoms, more particularly —CH₂—, —CH₂CH₂—, —CH₂CH₂CH₂—, —CH₂(CH₃)CH₂—, dodecyl, cetyl, or stearyl, preferably —CH₂CH₂—; and R⁴ is H or OH; preferably all of the R³ groups within any one Formula III molecule are the same; preferably all of the R⁴ groups within any one Formula III molecule are the same. In a preferred embodiment, R^{a} in Formula I and R^{c} in Formula II are the same so that manufacture thereof is easier and less costly; however, these need not be the same. Highly preferred salts include those where R^{b} is monoethanolammonium, diethanolammonium; and triethanolammonium. The ratio of FA to FPES is at least 5% by weight FA based on FA+FPES total weight to not more than about 90% by weight FA based on total FA+FPES weight, preferably at least 7.5% by weight FA, more preferably at least about 10% by weight FA, even more preferably at least about 15% FA, still even more preferably at least about 20% FA, yet even more preferably at least about 25% FA. Further preferred minimum amounts of FA relative (by weight) to total FA+FPES include at least about 30%, at least about 35%, at least about 40%, at least about 45%, at least about 49%, and at least about 50%. Furthermore, upper limits on the FA content relative (by weight) to the total FA+FPES content include not more than about 85% by weight, not more than about 80% by weight, not more than about 75% by weight, not more than about 70% by weight, not more than about 65% by weight, not more than about 60% by weight, not more than about 55% by weight, not more than about 51% by weight. An approximately 50/50 weight ratio of FPES and FA is also a preferred embodiment. Any range obtained by selecting a lower limit set forth above explicitly or those between the explicitly disclosed lower limits and an upper limit explicitly disclosed or selected from an upper limit between explicitly disclosed upper limits are considered to be explicitly disclosed herein and are suitable ranges. Thus for purposes of disclosure, a range of from 25% to 55% is considered disclosed as is a range of from 49% to 85% and all such other ranges. Furthermore, a range defined by two of the minimum amounts as well as a range defined by two of the maximum amounts is also considered disclosed. Thus, a range of 20% to 40% as well as a range of 55% to 85% and other similarly defined ranges are considered disclosed. It is a matter of routine for one of ordinary skill to select out two numbers from the explicitly disclosed minimums and maximums to define an appropriate range of the FA content relative to the total FA+FPES content by weight. Commercially available exemplary materials for the FPES component include, without limitation, C₈-C₁₈ perfluoroalkylethylphosphate diethanolamine salt (available form Dupont as Zonyl RP; from Asahi Glass Company as Asahi Guard AG-530; from Asahi Glass Company as Surflon S-112; MIC Specialty Chemicals as Repearl F-53; and Innovative Chemical Technologies as Flexiwet PD-30); C₆-C₁₈ perfluoroalkylethyl phosphate ammonium salt (available as Zonyl FSE, Zonyl FSP; or Zonyl FSE from DuPont; Flexiwet PAD-30 from Innovative Technologies; and Lodyne P-208E from Ciba). Commercially available materials for the FA component include, without limitation, C₆-C₁₂Perfluoroalkylethanol (available as Cheminox FA-N from NOK; as Zonyl BA from DuPont; as Fluowet EA-812 from Clariant, and is also available from Asahi Glass Company, Daikin, and Dainippon Ink and Chemicals. Esterifying the FA component material with phosphoric acid will yield the corresponding FPES components which may then be neutralized with the appropriate base to achieve the specific salts set forth herein.

Turning with greater specificity to the group R¹, it is preferably a substantially fluorinated uninterrupted C₆₋₁₈hydrocarbon, i.e., not more than 20%, more preferably not more than 10% of the carbon atoms of R¹ group are not fully fluorinated. R¹ may have up to 4 carbons (other than those that are members of the “interrupting functional groups” that are not totally fluorinated, but preferably have not more than two, still more preferably not more than 1 that are not completely fluorinated, and most preferably are perfluorinated (other than for the “interrupting heteroatoms and functional groups). In the most preferred embodiment, R¹ is a perfluorinated group without any interruptions from heteroatoms or functional groups.

The most preferred group of compounds for the FPES are of Formula IV

(PerfluoroC₆₋₁₈alkyl-(CH₂)₂O—)ₓP(O)(O^{-+N(H)}ᵥ(CH₂CH₂OH)₄₋ᵥ)_{y} IV

where x, y, and v are as defined above and the most preferred group of compounds for the FA are of Formula V

PerfluoroC₆₋₁₈alkyl-(CH₂)₂OH V.

An even more highly preferred subgroup are those of formula IV and V where the perfluoroC₆₋₁₈alkyl group is limited to perfluoroC₈₋₁₈alkyl. Less advantageous, but nonetheless useful embodiments are those of formula IV and V where the perfluoroC₆₋₁₈alkyl group is limited to perfluoroC₆₋₈alkyl (without substantial presence of larger perfluororoalkyl portions). In this latter embodiment (the perfluyoroalkyl being C₆₋₈), when used with ethanol as the solvent, the invention is limited to v being less than 4 (i.e. the NH₄ salt being excluded). It should be noted that this is not a disclaimer of compositions having the perfluoroalkyl being C₆₋₈ along with a significant or substantial amount of perfloroalkyls having larger numbers of carbon atoms as well.

In any particular product, the FPES component may be a single chemical entity or a mixture of FPESs. Typically the FPES is a blend of mono, di, and tri esters of the phosphoric acid and the FA is the same alcohol that participates in the esterification reaction. Thus, where one wishes to use a different FA than that used in the esterification, the esterification is done without molar excess of the FA, so that all of the FA can be used in the esterification. This allows for the addition of a different FA to complete the present invention. Alternatively, should one wish to use the same FA as in the esterification reaction, one can either use no molar excess in the esterification (adding more FA later), or use an excess amount of the FA in the esterification reaction so that the product of the esterification already contains a blend of FA and FPES. Where some intermediary amount of FA is used in the esterification reaction, additional amounts of the same FA or a different FA may also be used to bring the total FA content into the invention ranges, resulting in a blend of FAs in the various products.

The FPES components that have been made using perfluoralkyl-ethan-2-ol as the FA by reacting it with polyphosphoric acid are primarily monoesters with small amounts of the other esters, while the reaction of the FA with phosphorous pentoxide are primarily blends of mono ester and diester with small amounts of the others. These reactions and the products are known in the art and are conducted in the art in the absence of excess FA so that only a small percent of free FA remains in the product. Pure free FA has a substantially lesser foaming ability for organic solvents than does pure FPES or those with a minor amount (generally less than about 1%) which may result in the normal production in the art. In fact, because of this, the commercial manufacturers of FPES try to minimize the amount of free FA in their products and therefore conduct the reactions with as close to stoichiometric amounts of FA or less. These materials are described more fully in portions of US 2005-0129626, and compounds of Formula IV are available from Mason Chemical Company. Analogously, the various other FAs can be reacted with either polyphosphoric acid or phosphorous pentoxide to obtain the corresponding esters. Where desired, purification to isolate a single ester can be done, but is typically not commercially done since the blends work just about as well without substantially different properties being conferred by the pure single ester entities, whether mono ester, diester, or triester. One of the important features of the present invention is the surprising increase in foam stability without use of thickeners or the need for propellant or pressurized gas by merely increasing the FA content of these materials from the levels contained therein as a byproduct of the art manufacture of the esters to the invention range of from at least about 5 by weight up to not more than about 90% by weight (relative to the total FA+FPES content).

Having described the important components that go into the invention products and methods, typical such products will now be discussed.

When dealing with high alcoholic content formulations where the alcohol is ethanol or isopropanol, the present invention can be used in any of the fields discussed in this specification. Thus, the following discussion will be presented primarily with these two alcohols and with respect to the personal care product area, but should be understood as being applicable fully to all application areas discussed in this specification.

Concentrates of the present invention and for use in the present invention are those having at least about 5% of an FA (or blend of FAs) in combination with not more than 95% of an FPES (or blend of FPESs) relative to the total of the FA+FPES. These blends can be used as is or can be dissolved in a suitable solvent that is acceptable for the end use application. Since the concentrates will be used for foaming high organic solvent from the list set forth above, use of one of those solvents is a suggested choice, especially methanol, ethanol or isopropanol (where they are compatible with the end use application). A particularly useful concentrate formulation utilizes a solvent of 50% by weight methanol, 35% by weight water and 15% by weight solids. Alternatively, since the materials are produced in aqueous solution and the presence of some water is generally acceptable, an aqueous solution of the FA/FPES blend in amounts up to about 30% by weight of the blend in water may be used. Higher weight % s may be used if desired, but the solution becomes less stable at those higher levels. Still, if that is not of concern, they may be used. Concentrated solutions are only limited by whether the amount of water introduced into the final formulation is acceptable. Since the amount of FPES to be used in a commercial product may be as low as 0.01%, a concentrate formulation of the present invention may have as low as less than 0.1% solids content up to the maximum that is soluble or suspendable in the solvent of choice, or may be a dry blend of the FPES and FA in the correct proportions. Generally because the concentrates will be added to formulations that already have a high organic solvent content, it is desirable to use more highly concentrated concentrates (so as to keep the addition of further solvent down). However, this is to be balanced by the need to accurately measure how much of the concentrate to add in any particular application. Too highly concentrated concentrates are less desirable where a small amount is to be added to a small product batch so that there is difficulty in accurately measuring the correct amount or that leads to waste of costly materials. Thus, particular concentrations of concentrates will be suitably chosen depending on both the amount of material needed for foaming in a particular formulation and the size of the commercial or non-commercial batch involved. Those of ordinary skill in the art would be well aware of how to adjust these variables.

The various FAs used in the present invention are compounds which are commonly available or can be readily made. Perfluoroalkyl-carboxylic acids are readily available commercially and can be readily converted to perfluoroalkyl-carbonyloxy-alkylalcohol by reaction with an alkylene glycol. The carbonyl group of the perfluoroalkylcarboxy starting material can be hydrogenated to the corresponding alcohol and reacted with a hydroxylprotected hydroxyalkanoic acid to give perfluoroalkyloxycarbonyl-alkylalcohols (where the alcohol function is protected), which can then have the protected hydroxyl group deprotected. Similar chemistry can be utilized to form thioether linkages and amide linkages. Where the alkylene glycol or hydroxyl alkanoic acid is replaced by its fluorinated counterpart, the reactions result in the linkages being within the fluorinated portion of the molecule. Those or ordinary skill in the art will appreciate variations on this theme to obtain the other linkages mentioned herein.

Once the FA compounds are prepared, or obtained, they are reacted with either a polyphosphoric acid or phosphorous pentoxide. If the FA is present in excess relative to the polyphosphoric acid or phosphorous pentoxide, then the invention concentrates can result directly in the reaction vessel. Because the usual commercial FPES products try not to have free FA (the free FA being thought to negatively impact on the foaming ability of the FPES because they are not surfactants, solubilizers, or hydrotropes in their own right), the FPES compounds are made under conditions in the art which generally have less than 2% free FA, more usually less than about 1% free FA or no free FA at all. If the FA is not present in sufficient excess, then additional FA needs to be added to bring the free FA content up to the appropriate minimum weight %. When polyphosphoric acid is used, the FPES is primarily a monoester and the amount of FA in the reaction is based thereon so that to arrive at the correct FA to FPES ratio, one would use slightly more than 7.5% by weight of FA more than a molar equivalent of the polyphosphoric acid to reach a final content of just about 7.5% by weight FA out of the total of FA+FPES. Similarly, when using phosphorous pentoxide, primarily a mixture of mono ester and di-ester and as such, a larger amount of FA is needed in order to allow for the fact that a portion of the FA is bound up as diester. Regardless of which is used the polyphosphate or the phosphorous pentoxide and regardless of whether stoichiometric amounts of FA are used or excess FA is present, the end product can be brought up to the appropriate level of FA by subsequent addition of FA if needed. Alternatively, the additional FA can be added at the point in time when formulating a particular end use formulation.

Having discussed the concentrate and the manner of making it, the remainder of the discussion will be directed to various end uses for the FA/FPES combination and concentrate thereof. As previously stated, the present invention blend of FA and FPES and concentrated solutions thereof are useful in a multitude of applications that include the personal care product arena (including, but not limited to foamable hand sanitizers, foamable shampoos, foamable personal cleansers (such as shower or bath oil foams, and other foamable soaps), foamable brushless shave creams, sunscreen foams, foam make-up removers, foam analgesic rubs, antiperspirant foams, foam pet grooming products, etc.); medicated foams containing any number of suitably dispensed active agents (including, but not limited to foams for external use, internal use (generally in body cavities other than oral, but oral use may be suitable as well); foamed carriers that are solid and rapidly dissolvable, and loaded with an active agent, whether medicinal or not); household and industrial (hard surface or non-hard-surface) cleaning compositions (including, but not limited to foams that collapse on the application of pressure and foamed solid carriers loaded with an active agent which dissolve rapidly in water); industrial applications (including, without limitation, oilfield applications (such as foaming methods to open up “tight wells” in the production/recovery of natural gas—see, for example, U.S. Pat. No. 7,049,436), foam insulation, foam containers, and foam packaging products) to name a few.

In the remainder of this specification, unless specifically set out otherwise or dictated by the context as that would be understood by those of ordinary skill in the respective product field, all percentages are w/w %.

Personal Care Products

Hand sanitizers typically have high lower alcohol content, especially high content of either ethanol or isopropanol and foamable products in this product area are difficult to obtain without the use of thickeners that impart a tackiness to the body surfaces being sanitized. Omission of the thickeners in the past has led to foams (when foams could initially be obtained) that were thin and watery so that they would run off the body surfaces quickly, or foams that would collapse too readily. US 2005-0129626 proposed a means to obtain a foam which could avoid the thickeners, but still did not result in a foam that was as thick as desired and tended to run off the body surfaces too readily. The present invention used in a hand sanitizer formulation allows for obtaining a thick foam that does not run, that is stable, that collapses on pressure being applied, that does not have a tacky feel either when applied or afterwards, and that allows one to eliminate the thickeners and foam stabilizers previously used in this product line (however, if desired for added stability, one may include either or both of the thickeners and/or foam stabilizers).

A typical non-limiting hand sanitizer composition of the invention which incorporates the FA and FPES of the invention contains
(a) a lower alcohol of up to 4 carbons, preferably ethanol or isopropanol, more preferably, ethanol, in an amount of at least 40 v/v % (based on the entire composition), preferably about 50 v/v % to about 80 v/v %, more preferably about 60 v/v % to about 70 v/v %;
(b) the FA/FPES combination of the invention in a total amount of about 0.01 to about 2% w/w (solid content of FA/FPES relative to the entire sanitizer formulation);
(c) optionally a foam stabilizer, such as, without limitation, 1,3-butylene glycol and 2-butoxyethanol, etc., and combinations thereof, in amounts of up to about 5% each, preferably in an amount of from about 0.001% to about 3%;
(d) optionally one or more lipid layer enhancer, especially a mixture of an alkylglucoside and glyceryl oleate; and/or one or more moisturizers or humectants (i.e. glycerine, propylene glycol, etc.);
(e) water
(f) optional fragrance.

The alcohol based compositions may contain up to about 10% by weight of other active ingredients or additives or combinations thereof commonly added to aerosol compositions or to toiletries, cosmetics, pharmaceuticals, etc. Materials that may be added may include organic gums and colloids, lower alkanolamides of higher fatty acids, short chain diols and/or triols, alkylglucosides, fragrance, coloring matter, additional emollients, ultraviolet absorbers, solvents, emulsifiers, foam stabilizers or mixture of such stabilizers, suspending agents, buffers, conditioning agents, antioxidants, bactericides, medicinal active ingredient, and the like. Few, if any of these will be added to the hand sanitizer products of the invention, but the inclusion thereof is permissible and still within the present invention.

Typical shampoo, foamable shave cream, foamable bath oil, and foamable shower gel formulations of the present invention will include standard shampoo, shave cream, bath oil, and shower gel components, respectively, that are compatible with the high organic solvent content of the present invention along with the high organic solvent in the appropriate concentration and the FPES/FA components required by the present invention.

Typical sunscreens of the present invention will include water, an alcohol, the active agent combination of the present invention and one or more sunscreen active agents (generally selected (without limitation thereto) to sunscreens such as octylmethoxycinnamate, oxybenzone, etc and mixtures thereof). Fragrances may be optional ingredients as well as emollients known in the art.

Typical make-up removers (including nail polish remover) of the present invention will include standard make-up remover components that are compatible with the high organic solvent content of the present invention along with the high organic solvent in the appropriate concentration and the FPES/FA components required by the present invention.

Typical analgesic rubs of the present invention will include analgesic active agents typical of analgesic rubs that are compatible with the high organic solvent content of the present invention along with that organic solvent and the FPES and FA required by the present invention.

Typical antiperspirants of the present invention will include typical antiperspirants that are compatible with the high organic solvent content of the present invention along with that organic solvent and the FPES and FA required by the present invention.

Still other personal care products that can be formulated with the present invention include, without limitation, skin toners, pore opening skin cleansers, biosilk silk hair treatments, skin clearing complexes, body spritzes, firm hold hair sprays, etc. Typical pet grooming products of the present invention will include typical pet grooming active agents that are compatible with the high organic solvent content of the present invention along with that organic solvent and the FPES and FA required by the present invention. In certain highly preferred embodiments, the invention products are substantially free, if not totally free of materials such as polyethyleneglycols, and/or emollient materials.

Pharmaceutical and Veterinary Products

Medicinal foams include those in which the foam carries an active agent for application to the body, whether internal (as an ingestible foam or otherwise) or external (as in a foaming external application product such as a CALADRYL® clear foam), as well as those products in which a foam carries the active agent and the foam structure and active agent is transformed into a hardened open network lattice, much like spun sugar is a loose connection of solid sugar strands. Where the hardened lattice is one of materials that are readily soluble, the structure can be used to deliver medication in a rapidly dissolving format with the presence of a minimal amount of water. Thus, such formulations can be used to administer medication in a tablet format when water is not available or when the patient cannot adequately swallow tablet medication. In addition, with other materials used for the hardened structure of the lattice, highly porous drug laden forms can also be prepared which do not dissolve, but which can deliver the drug laden therein through diffusional processes and therefore may be suitable for transdermal application, buccal or vaginal application, and where the structure dissolves slowly or not at all, implants, etc. Note that in the case of these hardened lattices, the high alcoholic content of the foam is driven off in the process of hardening the foam into the lattice structure so that application to mucous membranes is not of concern. One manner of creating the hardened open lattice forms is to create the appropriate foam via the present invention and subject the foam to a flash freezing with subsequent vacuum drying off of the solvent.

However, the main application of the present invention in the medication arena is the delivery of medicinals through a foam that is applied as a foam to the body and which upon the application of pressure in some fashion collapses the foam. This is especially useful in applying medicinals to the skin surface. A stable foam allows one to apply the foam and spread it without the foam running off too rapidly. A foam which collapses upon pressure application assures that the medication is rapidly presented to the surface in question.

A typical topical medicinal foam is one that is analogous to the high alcoholic content hand sanitizers except that a medicinal agent is included in an appropriate concentration, and the alcohol content can be significantly below 60%, and may be below about 50%, 45%, 40%, 35%, 30%, or even 25%.

A fast dissolving tablet made from hardened lattice may be prepared in any number of ways with the most direct use of the invention being to create a solution of the desired formulation in one of the solvents that is foamable within the present invention; create a foam from the active agent containing solution; and freeze dry the foam. Other manners of creating these kinds of tablets will be apparent to those of ordinary skill in the art.

An erodible matrix tablet made from hardened lattice can be made in the same manner as the fast dissolving tablet but using matrix materials that create a denser foam (or merely are not quite so soluble so that the erosion of the matrix once ingested is at a slower rate) or by using a lesser amount of the FPES/FA blend or that utilizes an extreme ratio between the FPES and FA so that foaming is not as great and a denser product results.

A non-erodible diffusion release matrix tablet made from hardened lattice in substantially the same way as an erodible tablet is made except that the material forming the erodible material is either replaced entirely or substantially by materials that are not erodible in the environment of use.

An implant matrix made from hardened lattice in the same manners as set forth above for the tablets described above, the only real distinction being that the product is implanted rather than being ingested.

Household Cleaners

Household cleaners can be made using the present invention in a number of ways as well. Foams themselves as household cleaners may include cleaning agents such as soaps and detergents and may be of high alcoholic content. Additionally, solid, fast dissolving products may be made using the hardened lattice technology so that substantial weight and space may be conserved in the manufacture, distribution, and storage of products, without substantial problems in subsequent dissolution of the solids are enabled with the present invention. Still further, time release erodible and non-erodible diffusion systems of cleaning agents in the context of toilet bowl cleaners, water purification systems, etc. are also possible.

Typical foam household cleaners (include bathroom and kitchen cleansers, such as tub, tile, sink, and toilet cleansers), foam laundry products (which typically are applied to clothes either when laundering is not convenient or slightly before or at the time of laundering), foam carpet cleaners, foam fabric deodorizers (such as a foamable version of products like FABREEZE®), vehicle exterior surface cleansers, etc. will typically contain components known in the art for those applications that are compatible with the high organic solvent of the invention along with the organic solvent in question and the FPES and FA required by the present invention. Typical, non-limiting formulations (in the art) for some of these applications can be found in US 2002-0160924 and US 2002-0187908, incorporated by reference herein. The formulations disclosed in these references can readily be modified for use in the present invention.

Typical fast dissolving laundry detergent tablets can be prepared in manners analogous to those for the fast dissolving oral matricies discussed above except that the components other than the FPES, FA, and the organic solvent are those typically found in the laundry area.

Industrial Hard Surface Cleaners

Industrial hard surface cleaners include products that are analogous to those in the household cleaner category but which are more concentrated or cover a wider range of soils needing to be cleaned. In addition, there are many other types of surfaces that require cleaning in the industrial setting in which high alcoholic solutions are needed and foamable products are desired, but the presence of thickeners and/or stabilizers are either problematic or completely unacceptable. In many of these contexts the alcohol is actually an active solvent for the material intended to be removed so that the higher alcoholic content is highly advantageous. Exemplary types of such cleaners include acid degreasers and cleaners for printing equipment. Other highly advantageous applications include cleaners in the electronic component area where it is frequently necessary to clean very precise areas of certain electronic components without exposing other areas to the cleaners. In such cases stable foams allow for precise placement of the cleaner, either directly to the substrate to be cleaned or by application of the foam to an applicator and then precise area cleaning.

Other Industrial Applications

Still other application fields for the present invention include the production of foamed materials where the foam is hardened in place to produce foam insulation, foam containers and foam packaging products with subsequent evaporation of the lower alcohol(s) used. This particular application allows for the use of a wider range of polymeric materials that will be foamed and polymerized in place than those prepared from strictly aqueous foams and also allows production of these articles using current polymers and monomers without the use of more environmentally hazardous solvent materials.

The compositions may also include an antimicrobial agent. This is particularly useful in the personal care products, but may be desirable in other formulations as well.

The following antimicrobials are offered as non-limiting examples of suitable antimicrobials for use in the present invention and may include iodine, a complexed form of iodine, parachlorometaxylenol, triclosan, hexachlorophene, a phenol, silver, a silver salt, silver oxide, copper, copper salts, copper oxide, and mixtures thereof.

Moisturizes and/or emollients may also be desired in the personal care product of the invention and suitable, non-limiting, examples thereof for use in the present formulations include lanolin, vinyl alcohol, polyvinyl pyrrolidone and polyols selected from the group consisting of glycerol, propylene glycol, glyceryl oleate and sorbitol, cocoglucoside or a fatty alcohol selected from the group consisting of cetyl alcohol, stearyl alcohol, lauryl alcohol, myristyl alcohol and palmityl alcohol, cetyl alcohol, ceteareth 20, and combinations thereof, present in an amount up to about 5%.

The compositions formulated to be dispensed as a foam need not be used with pressurized gas or propellant. However, for many product presentment situations, pressurized gas or propellant formats are desired. There is nothing in the present invention which prevents the use of the invention compositions with such pressurized gas or propellants. Thus, an aerosol propellant in an amount from about 3 to about 20 weight percent of the total composition may be used for pressurized discharge of the foam. The aerosol propellant may include propane, carbon dioxide, butane, dichloro difluoro methane, dichloro tetra fluoro ethane, octafluorocyclo butane; 1,1,1,2-tetrafluoroethane; 1,1,1,2,3,3,3 heptafluoropropane, and 1,1,1,3,3,3,-hexafluoropropane. When stored in a metal container with propellant, the formulation may include a corrosion inhibitor such as sorbic acid, benzoic acid, potassium sorbate and sodium benzoate, in an amount from about 0.1 to about 5 weight percent of the total composition.

### EXAMPLES

The following examples exemplify, but do not limit, the present invention. Masurf AF-110DE is a 1:1 blend of 2-(C₈-C₁₈ perfluoroalkyl)ethanol with the diethanolamine salts of 2-(C₈-C₁₈ perfluoroalkyl)ethyl esters of phosphoric acid in ethanol having 10 wt % of the blend as active agents. Masurf AF-115DE is a 1:1 blend of 2-(C₈-C₁₈ perfluoroalkyl)ethanol with the diethanolamine salts of 2-(C₈-C₁₈ perfluoroalkyl)ethyl esters of phosphoric acid in ethanol having 15 wt % of the blend as active agents. Masurf AF-210AE is a 1:1 blend of 2-(C₈-C₁₈ perfluoroalkyl)ethanol with the ammonium salts of 2-(C₈-C₁₈ perfluoroalkyl)ethyl esters of phosphoric acid in ethanol having 10 wt % of the blend as active agents. Masurf AF-115AE is a 1:1 blend of 2-(C₈-C₁₈ perfluoroalkyl)ethanol with the ammonium salts of 2-(C₈-C₁₈ perfluoroalkyl)ethyl esters of phosphoric acid in ethanol having 15 wt % of the blend as active agents. Masurf AF-410TE is a 1:1 blend of 2-(C8-C18 perfluoroalkyl)ethanol with the triethanolamine salts of 2-(C8-C18 perfluoroalkyl)ethyl esters of phosphoric acid in ethanol having 10 wt % of the blend as active agents. Masurf AF-610E is a 1:1 blend of 2-(C8-C18 perfluoroalkyl)ethanol with 2-(C8-C18 perfluoroalkyl)ethyl esters of phosphoric acid in ethanol having 10 wt % of the blend as active agents.

### Example 1

A hand sanitizer is formulated as follows:

A

| | | |
|---|---|---|
| Water | 27.8 | wt % |
| Masurf AF-410TE | 5 | wt % |
| SD Alcohol 40 | 62 | wt % |
| Fragrance | 0.1 | wt % |
| Glycereth-2-cocoate | 0.1 | wt % |
| Butylene glycol | 5 | wt % |

B

| | | |
|---|---|---|
| Water | 27.8 | wt % |
| Masurf AF-115AE | 5 | wt % |
| SD Alcohol 40 | 62 | wt % |
| Fragrance | 0.1 | wt % |
| Glycereth-2-cocoate | 0.1 | wt % |
| Butylene glycol | 5 | wt % |

### Example 2

A skin toner is formulated according to the formulation below:

A

| | | |
|---|---|---|
| Water | 51.2 | wt % |
| Masurf AF-110DE | 3 | wt % |
| SD Alcohol 40 | 45 | wt % |
| Glycerin | 0.5 | wt % |
| Panthenol | 0.1 | wt % |
| DMDM Hydantoin | 0.1 | wt % |
| Polysorbate 20 | 0.1 | wt % |

B

| | | |
|---|---|---|
| Water | 51.2 | wt % |
| Masurf AF-115DE | 3 | wt % |
| SD Alcohol 40 | 45 | wt % |
| Glycerin | 0.5 | wt % |
| Panthenol | 0.1 | wt % |
| DMDM Hydantoin | 0.1 | wt % |
| Polysorbate 20 | 0.1 | wt % |

### Example 3

A pore opening skin cleanser of the invention is formulated as follows:

A

| | | |
|---|---|---|
| Water | 36.65 | wt % |
| Masurf AF-110DE | 3 | wt % |
| SD Alcohol 40 | 60 | wt % |
| Glycolic Acid | 0.1 | wt % |
| Ammonium glycolate | 0.1 | wt % |
| Sorbitol | 0.1 | wt % |
| Eucalyptus oil | 0.05 | wt % |

B

| | | |
|---|---|---|
| Water | 36.65 | wt % |
| Masurf AF-115DE | 3 | wt % |
| SD Alcohol 40 | 60 | wt % |
| Glycolic Acid | 0.1 | wt % |
| Ammonium glycolate | 0.1 | wt % |
| Sorbitol | 0.1 | wt % |
| Eucalyptus oil | 0.05 | wt % |

### Example 4

A biosilk silk hair treatment is formulated as follows:

A

| | | |
|---|---|---|
| Water | 24.4 | wt % |
| Masurf AF-110DE | 5 | wt % |
| SD Alcohol 40 | 50 | wt % |
| Cylomethicone | 10 | wt % |
| Dimethicone | 10 | wt % |
| Panthenol | 0.5 | wt % |
| Ethyl ester of hydrolyzed silk | 0.1 | wt % |

B

| | | |
|---|---|---|
| Water | 24.4 | wt % |
| Masurf AF-115DE | 5 | wt % |
| SD Alcohol 40 | 50 | wt % |
| Cylomethicone | 10 | wt % |
| Dimethicone | 10 | wt % |
| Panthenol | 0.5 | wt % |
| Ethyl ester of hydrolyzed silk | 0.1 | wt % |

### Example 5

An SPF 20 sunscreen is formulated as follows:

A

| | | |
|---|---|---|
| Water | 34.55 | wt % |
| Masurf AF-410TE | 5 | wt % |
| SD Alcohol 40 | 60 | wt % |
| Dimethicone | 0.1 | wt % |
| Octylmethoxycinnamate | 0.1 | wt % |
| Oxybenzone | 0.1 | wt % |
| Sodium PCA (sodium | 0.1 | wt % |
| pyrrolidone carboxylic acid) | | |
| Allantoin | 0.05 | wt % |

B

| | | |
|---|---|---|
| Water | 34.55 | wt % |
| Masurf AF-115DE | 5 | wt % |
| SD Alcohol 40 | 60 | wt % |
| Dimethicone | 0.1 | wt % |
| Octylmethoxycinnamate | 0.1 | wt % |
| Oxybenzone | 0.1 | wt % |
| Sodium PCA (sodium | 0.1 | wt % |
| pyrrolidone carboxylic acid) | | |
| Allantoin | 0.05 | wt % |

### Example 6

A skin clearing complex is formulated as follows:

A

| | | |
|---|---|---|
| Water | 38.8 | wt % |
| Masurf AF-110DE | 5 | wt % |
| SD Alcohol 40 | 48 | wt % |
| Salicylic Acid | 5 | wt % |
| Ammonium lactate | 3 | wt % |
| Lactic Acid | 2 | wt % |
| Retinol | 0.1 | wt % |
| Retinyl palmitate | 0.1 | wt % |

B

| | | |
|---|---|---|
| Water | 38.8 | wt % |
| Masurf AF-115DE | 3 | wt % |
| SD Alcohol 40 | 48 | wt % |
| Salicylic Acid | 5 | wt % |
| Ammonium lactate | 3 | wt % |
| Lactic Acid | 2 | wt % |
| Retinol | 0.1 | wt % |
| Retinyl palmitate | 0.1 | wt % |

### Example 7

A body spritz is formulated as follows:

A

| | |
|---|---|
| Water | 46.6 wt % |
| Masurf AF-110DE | 3 wt % |
| SD alcohol 40 | 50 wt % |
| Sodium PCA | 0.1 wt % |
| Extract (Botanical) | 0.1 wt % |
| Aloe Barbadensis | 0.1 wt % |
| Fragrance | 0.1 wt % |

B

| | |
|---|---|
| Water | 46.6 wt % |
| Masurf AF-115DE | 3 wt % |
| SD alcohol 40 | 50 wt % |
| Sodium PCA | 0.1 wt % |
| Extract (Botanical) | 0.1 wt % |
| Aloe Barbadensis | 0.1 wt % |
| Fragrance | 0.1 wt % |

### Example 8

A firm hold hair spray is formulated as follows:

A

| | |
|---|---|
| Water | 32.1 wt % |
| Masurf AF-110DE | 4 wt % |
| SD alcohol 40 | 60 wt % |
| Acrylates copolymer | 1 wt % |
| Acrylates/octyl acryl- | 1 wt % |
| Amide copolymer | |
| Polyurethane-1 | 1 wt % |
| Dimethicone copolyol | 0.5 wt % |
| Aminomethyl propanol | 0.3 wt % |
| Phytantriol | 0.1 wt % |

B

| | |
|---|---|
| Water | 32.1 wt % |
| Masurf AF-115DE | 4 wt % |
| SD alcohol 40 | 60 wt % |
| Acrylates copolymer | 1 wt % |
| Acrylates/octyl acryl- | 1 wt % |
| Amide copolymer | |
| Polyurethane-1 | 1 wt % |
| Dimethicone copolyol | 0.5 wt % |
| Aminomethyl propanol | 0.3 wt % |
| Phytantriol | 0.1 wt % |

### Example 9

A CALADRYL® foam is formulated as follows:

A

| | |
|---|---|
| Water | 34.6 wt % |
| Masurf AF-110DE | 4 wt % |
| SD alcohol 40 | 60 wt % |
| Fragrance | 0.1 wt % |
| Pramoxime HCl | 1 wt % |
| Zinc oxide | 0.1 wt % |
| Camphor | 0.1 wt % |
| Glycerin | 0.1 wt % |

B

| | |
|---|---|
| Water | 34.6 wt % |
| Masurf AF-115DE | 4 wt % |
| SD alcohol 40 | 60 wt % |
| Fragrance | 0.1 wt % |
| Pramoxime HCl | 1 wt % |
| Zinc oxide | 0.1 wt % |
| Camphor | 0.1 wt % |
| Glycerin | 0.1 wt % |

### Example 10

A printing equipment cleaner formulation is formulated as follows:

A

| | |
|---|---|
| Water | 40 wt % |
| Masurf AF-210AE | 5 wt % |
| Acetone | 45 wt % |
| Isopropyl acetate | 10 wt % |

B

| | |
|---|---|
| Water | 40 wt % |
| Masurf AF-115DE | 5 wt % |
| Acetone | 45 wt % |
| Isopropyl acetate | 10 wt % |

### Example 11

Two methanol foamer formulations are formulated as follows:

A1

| | |
|---|---|
| Water | 58 wt % |
| Masurf AF-110DE | 2 wt % |
| Methanol | 40 wt % |

A2

| | |
|---|---|
| Water | 8 wt % |
| Masurf AF-110DE | 2 wt % |
| Isopropyl acetate | 90 wt % |

B1

| | |
|---|---|
| Water | 58 wt % |
| Masurf AF-115DE | 2 wt % |
| Methanol | 40 wt % |

B2

| | |
|---|---|
| Water | 8 wt % |
| Masurf AF-115DE | 2 wt % |
| Isopropyl acetate | 90 wt % |

### Example 12

An acid degreaser is formulated as follows

A

| | |
|---|---|
| Water | 44.5 wt % |
| Masurf AF-610E | 5 wt % |
| Ethylene glycol- | 50 wt % |
| Monobutyl ether | |
| Phosphoric acid | 0.5 wt % |

B

| | |
|---|---|
| Water | 44.5 wt % |
| Masurf AF-115DE | 5 wt % |
| Ethylene glycol- | 50 wt % |
| Monobutyl ether | |
| Phosphoric acid | 0.5 wt % |

### Examples 13-24

Each of examples 1-12 is repeated except that Masurf AF-115AE is used in place of Masurf AF-115DE

### Examples 25-72

Each of Examples 1-24 is repeated except that an analog of the Masurf material having the ratio of phosphate ester salt to perfluoroalkylethanol set forth in the following table is used:

| | Examples 25-28 | Example 49-72 |
|---|---|---|
| Perfluoro alkylethyl | 10 parts by weight | 85 parts by weight |
| phosphate ester salt | | |
| perfluoroalkylethanol | 90 parts by weight | 15 parts by weight |

### Example 73

### Qualitative and Quantitative Foam Assessment

In the qualitative test, a 62% (w/w %) ethanol solution in water is prepared with the FA, FPES, or both FA and FPES using the acid form of the FPES or one of the 7 salts thereof as indicated in the table below so that the composition contained 0.375% w/w % of the total of the fluorinated compounds in any particular formulation. The compositions were delivered through an AirSpray International pumpfoam nozzle onto a glass plate (approximately 0.6 grams/6 cc of foam for each test). The foams were evaluated for foam stability and the results are in the table as NF (no foam, foam breaks instantly), FB (fast breaking foam, foam breaks in less than 30 seconds), MB (medium breaking, foam breaks between 20 and 120 seconds) and SF (stable foam, foam is stable for more than 10 minutes).

In the quantitative test, 50 ml of the solutions are placed in a stoppered 250 ml graduated cylinder, which is then shaken for 30 seconds. Foam heights are recorded at 1 minute (total foam height in column) and at five minutes (total height of the foam in the column, inclusive of liquid layer below and a separate measure of the height of the liquid layer below the foam, the difference being the actual foam height in the column.

| | | | | | C₈₋₁₈Perfluoroalkylethyl | | | |
|---|---|---|---|---|---|---|---|---|
| | | | | | phosphate (FPES) + | | | |
| | C₈₋₁₈Perfluoroalkylethyl | | | | C₈₋₁₈Perfluoroalkylethanol | | | |
| | phosphate (FPES) | | | | (FA) (0.375% | | | |
| | (0.375% active | | | | combined FPES and FA | | | |
| | FPES) | | | | (1:1)) | | | |
| | | | 5 min | 5 min | | | 5 min | 5 min |
| | | | (total)/ | Actual | | | (total)/ | Actual |
| Salt Used | Qual | 1 min | liquid | foam | Qual | 1 min | liquid | foam |
| Acid | NF | 50 | 50/50 | 0 | SF | 105 | 105/49 | 56 |
| Sodium | NF | 50 | 50/50 | 0 | SF | 105 | 105/49 | 56 |
| Ammonium | FB | 60 | 51/50 | 1 | SF | 110 | 110/48 | 62 |
| Monoethanolamine | MB | 110 | 76/42 | 34 | SF | 120 | 120/42 | 78 |
| Diethanolamine | MB | 110 | 90/42 | 48 | SF | 130 | 120/42 | 78 |
| Triethanolamine | MB | 104 | 98/44 | 54 | SF | 130 | 130/42 | 88 |
| Dimethylaminopropropylamine | NF | 51 | 50/50 | 0 | SF | 84/ | 84/45 | 39 |
| C₁₂alkyl dimethylamine | NF | 50 | 50/50 | 0 | MB | 110 | 80/46 | 34 |

| | |
|---|---|
| C₈₋₁₈Perfluoroalkylethanol (FA) | Qual = NF; 1 = min = 50; 5 min = total 50/liquid 50; |
| (0.375%) alone | 5 min actual foam = 0 |

| | | | | | C₆Perfluoroalkylethyl phosphate | | | |
|---|---|---|---|---|---|---|---|---|
| | | | | | (FPES) + | | | |
| | | | | | C₆Perfluoroalkylethanol (FA) | | | |
| | C₆Perfluoroalkylethyl phosphate | | | | (3.75% combined FPES and FA | | | |
| | (FPES) (3.75% active FPES) | | | | (1:1)) | | | |
| | | | 5 min | 5 min | | | 5 min | 5 min |
| | | | (total)/ | Actual | | | (total)/ | Actual |
| Salt Used | Qual | 1 min | liquid | foam | Qual | 1 min | liquid | foam |
| Ammonium | NF | 50 | 50/50 | 0 | NF | 50 | 50/50 | 0 |
| Diethanolamine | NF | 50 | 50/50 | 0 | FB | 60 | 54/50 | 4 |
| Triethanolamine | NF | 50 | 50/50 | 0 | FB | 64 | 58/50 | 8 |

| | |
|---|---|
| C₆Perfluoroalkylethanol (FA) | Qual = NF; 1 min = 50; 5 min = total 50/liquid 50; |
| (3.75%) alone | 5 min actual foam = 0 |

As can be seen from the above data the FA component has no foaming capability for high alcohol content formulations. Thus, it would not be expected to improve the alcohol foaming capabilities of the FPES when added to the FPES, and certainly one would have expected a negative effect when replacing a portion of the FPES with the FA component. Surprisingly, the inclusion of the FA in the ratio and amounts indicated by the present invention results in a significant improvement in foaming ability and foam stability for all of the salts and the free acid of the FPES when the FPES is the C₈₋₁₈perfluoroalkylethylphosphate ester and the FA component is C₈₋₁₈perfluoroalkylethanol. The test results also show surprising improvements in foaming ability and stability when the FPES is C₆ perfluoroalkylethylphosphate ester (diethanolamine salt or triethanolamine salt) and the FA component is C₆perfluoroalkylethanol.

Surface tension was also measured for the compositions having 62 w/w % ethanol in water and the FPES or FA or mixture in the solids concentration as indicated in the table below. The surface tension of the resulting composition is set forth in the table. The water content is adjusted to account for the presence or absence of the solids dissolved therein. The FPES compound is the diethanolamine salt of C₈₋₁₈Perfluoroalkylethyl phosphate and the FA component is C₈₋₁₈Perfluoroalkylethanol.

| | A | B | C | D | E | F | G | H |
|---|---|---|---|---|---|---|---|---|
| Ethanol | 62.0 | 62.0 | 62.0 | 62.0 | 62.0 | 62.0 | 62.0 | 62.0 |
| Water | 38.0 | 37.25 | 37.25 | 37.25 | 37.925 | 37.85 | 37.9625 | 37.925 |
| FPES | | | 0.75 | 0.375 | 0.075 | 0.075 | 0.0375 | 0.0375 |
| FA | | 0.75 | | 0.375 | | 0.075 | | 0.0375 |
| Surface tension | 27.4 | 27.4 | 22.8 | 22.8 | 26.5 | 26.5 | 27.1 | 27.1 |
| (dynes/cm) | | | | | | | | |

surface tension is highest for a formulation in the absence of surfactant and is reduced with increasing surfactant concentration until a maximum reduction in surface tension is reached. Addition of further amounts of surfactant do not reduce surface tension further. Foaming ability is generally better with lower surface tension than with higher surface tension. Formulation A in the table above is simply 62% w/w aqueous ethanol and has a tension of 27.4 dynes/cm. A comparison of formulations A and B, C and D, E and F, and G and H, show that the addition of the FA component to the formulation otherwise not having the FA component does not change the surface tension at all, so that one would also not expect the addition of the FA component to change the foaming capability or foam stability as compared to its parallel formulation absent the FA component. Comparison of formulation C and D further show that the maximum surface tension reduction has already been reached at 0.375% of FPES component, since the FA component is formulation D doesn't impact the surface tension (comparison of formulations A and B) and the surface tension is the same as between formulation C having 0.75% FPES and formulation D having 0.375% FPES. Thus, one would expect the FA component to not materially change the foaming properties of the formulations having the FPES but not the FA component. Surprisingly, as clearly demonstrated by the Qualitative and Quantitative data in the previous tables, the addition of the FA component to an FPES containing high alcohol content solution to reach the requirements of the present invention, substantially and unexpectedly improves the foaming characteristics of the formulation.

1. A method of foaming a high organic solvent containing formulation comprising incorporating into said high organic content formulation
(a) a fluorinated phosphate ester surfactant (“FPES”) of the formula
(R^{a}—O)ₓ—P(O)—(OR^{b})_{y} in combination with
(b) a fluorinated alcohol (“FA”) of the formula R^{c}OH,
where x is 1 to 3,
y is (3−x),
R^{a} is a group R¹-R²- with R¹ being a fluorinated C₆₋₁₈hydrocarbon optionally interrupted by groups of one or two non-fluorinated (or less fluorinated than in the rest of R¹ exclusive of the optional interruptions) carbons per interruption, and/or optionally interrupted by heteroatoms or functional groups,
R² is a lower alkylene group;
R^{b} is selected from H, alkali metal and positively charged amines, and
R^{c} is, independently of R^{a}, selected from the same group as R^{a} or may also be a shorter carbon chain version thereof;
wherein said FPES and said FA are present in a weight ratio of from a ratio of about 5 parts by weight FA to 95 parts by weight FPES (on the basis of FPES and FA only) to a ratio of about 90 parts by weight FA to about 10 parts by weight FPES (on the basis of FPES and FA only); and
(c) optionally water.

2. The method of claim 1 wherein said FPES and said FA are present just prior to foaming said formulation and the combined FPES and FA is present in an amount of at least 0.01 wt % based on the entire composition being foamed.

3. The method of claim 1 wherein the organic solvent that is to be foamed is selected from selected from the group consisting of lower aliphatic alcohol; C₅₋₆cyclic saturated or unsaturated alcohols; 5-6 membered cyclic saturated or unsaturated solvents having an oxygen ring member; lower alkyl-C(O)-lower alkyl; lower alkyl-O—C(O)-lower alkyl; lower alkylene glycols; and lower alkylene glycol-mono lower alkyl ethers and mixtures thereof.

4. The method of claim 3 wherein said solvent is selected from the groups consisting of methanol, ethanol, isopropanol, acetone, and isopropyl acetate.

5. A concentrate suitable as an additive for incorporation into a formulation for foaming comprising
(a) a fluorinated phosphate ester surfactant (“FPES”) of the formula
(R^{a}—O)ₓ—P(O)—(OR^{b})_{y} in combination with
(b) a fluorinated alcohol (“FA”) of the formula R^{c}OH,
where x is 1 to 3,
y is (3−x),
R^{a} is a group R¹-R²- with R¹ being a fluorinated C₆₋₁₈hydrocarbon optionally interrupted by groups of one or two non-fluorinated (or less fluorinated than in the rest of R¹ exclusive of the optional interruptions) carbons per interruption, and/or optionally interrupted by heteroatoms or functional groups,
R² is a lower alkylene group;
R^{b} is selected from H, alkali metal, and positively charged amines, and
R^{c} is, independently of R^{a}, selected from the same group as R^{a} or may also be a shorter carbon chain version thereof;
wherein said FPES and said FA are present in a weight ratio of from a ratio of about 5 parts by weight FA to 95 parts by weight FPES (on the basis of FPES and FA only) to a ratio of about 90 parts by weight FA to about 10 parts by weight FPES (on the basis of FPES and FA only); and
(c) optionally water.

6. A foamable composition comprising an active agent for the intended purpose, optionally additional excipients compatable with the end use of the composition and as an agent for making the composition foamable
(a) a fluorinated phosphate ester surfactant (“FPES”) of the formula
(R^{a}—O)ₓ—P(O)—(OR^{b})_{y} in combination with
(b) a fluorinated alcohol (“FA”) of the formula R^{c}OH,
where x is 1 to 3,
y is (3−x),
R^{a} is a group R¹-R²- with R¹ being a fluorinated C₆₋₁₈hydrocarbon optionally interrupted by groups of one or two non-fluorinated (or less fluorinated than in the rest of R¹ exclusive of the optional interruptions) carbons per interruption, and/or optionally interrupted by heteroatoms or functional groups,
R² is a lower alkylene group;
R^{b} is selected from H, alkali metal, and positively charged amines, and
R^{c} is, independently of R^{a}, selected from the same group as R^{a} or may also be a shorter carbon chain version thereof;
wherein said FPES and said FA are present in a weight ratio of from a ratio of about 5 parts by weight FA to 95 parts by weight FPES (on the basis of FPES and FA only) to a ratio of about 90 parts by weight FA to about 10 parts by weight FPES (on the basis of FPES and FA only); and
said foamable composition optionally containing water.

7. The foamable composition of claim 6 which is a product selected from the group consisting of those designed for personal care, medicinal use, veterinary use, pet grooming products, household cleaners and deodorizers, and industrial cleaners.

8. The foamable composition of claim 7 which is a hand sanitizer, a shampoo, a skin toner, a pore opening composition, a hair treatment composition, a sunscreen, a skin clearing complex, a body spritz, an aftershave, a cologne, a perfume, a deodorant, an antiperspirant, a make-up remover, a nail polish remover, a shave cream, a bath oil, a shower gel, an analgesic rub, a pet grooming composition, a veterinary medicinal composition, a pharmaceutical composition, a kitchen cleaner, a bathroom cleaner, a laundry product, or a vehicle exterior cleaner.

9. The composition of claim 7 which is an acid degreaser, a printing equipment cleaner, an electronic component cleaner, or a product used in the recovery of oil or natural gas from a subterranean oil and/or natural gas deposit.

10. The composition of claim 1 wherein said FPES is selected from C₈₋₁₈perfluoroalkylethylphosphate esters, being the mono-ester, di-ester, or tri-ester thereof, said mono- and di-esters, optionally having the non-esterified portions as free groups or salts thereof and the corresponding C₆perfluoroalkylethylphosphate mono-, di- and tri-esters and their salts, or mixtures thereof.

11. The composition of claim 1 wherein the FA component is selected from C₈₋₁₈perfluoroalkylethanol and C₆perfluoroalkylethanol, or mixtures thereof.

12. The composition of claim 1 wherein the FPES component is present as a free acid or in a salt form or a mixture thereof, wherein the salt form has a cation selected from the group consisting of sodium, potassium, ammonium, monoethanolammonium, diethanolammonium, triethanolammonium, dimethylaminopropylamine, C₁₂alkyldimethylammonium and mixtures thereof.

13. A foamable aqueous alcohol formulation comprising:
a) at least 60% w/w ethanol;
b) at least 0.01% of a fluorochemical blend, said fluorochemical blend comprising at least 5% w/w to not more than about 90% w/w based on the total fluorochemical blend of a fluoroalkylalkanol (FA) and a fluoroalkylalkanolphosphate mono-, di-, or tri ester or mixtures of said esters (FPES), said FA and FPES being of the formulas set forth in claim 1.

14. The foamable composition of claim 13 comprising
a) at least 60% w/w ethanol;
b) at least 0.01% of a fluorochemical blend, said fluorochemical blend comprising at least 5% w/w to not more than about 90% w/w based on the total fluorochemical blend of a fluoroalkylalkanol (FA) and a fluoroalkylalkanolphosphate mono-, di-, or tri ester or mixtures of said esters (FPES), said FA being a C₈₋₁₈perfluoroalkylethanol and said FPES being a C₈₋₁₈perfluoroalkylethylphosphate ester selected from mono-, di-, and tri-esters and mixtures thereof, each in free or salt form or mixtures thereof, wherein said salt is selected from the group consisting of the ammonium salt, the diethylammonium salt, the triethylammonium salt, and mixtures thereof.
